(19) United States
(12) Patent Application Publication
(10) Pub. No.: US 2015/0051625 A1
(43) Pub. Date: Feb. 19, 2015

(54) Treating Occlusions within Body Vessels

(71) Applicants: Gary PETRUCCI, Long Lake, MN (US); Puneet K. KHANNA, Rancho Mirage, CA (US)

(72) Inventors: Gary Petrucci, Long Lake, MN (US); Puneet K. Khanna, Rancho Mirage, CA (US)

(21) Appl. No.: 14/383,235

(22) PCT Filed: Mar. 5, 2013
(86) PCT No.: PCT/US13/29153

§ 371 Date: Sep. 5, 2014

Related U.S. Application Data
(60) Provisional application No. 61/606,655, filed on Mar. 5, 2012.

(51) Int. Cl. A61B 17/3207

(52) U.S. Cl. CPC A61B 17/320725; A61B 2017/320741
USPC 606/159

(57) ABSTRACT

A system for treating occlusions present within a body vessel. The device comprises a plaque-treating device having an elongate shaft and a distal plaque-treating portion adapted to remove hardened plaque from within a body vessel. The device further comprises a multi-balloon positioning catheter with a lumen into and through which lumen the plaque-treating device may be extended, the positioning catheter having at least three inflatable balloons positioned circumferentially about the lumen to controllably stabilize the lumen and its distal opening at a desired location within the body vessel, thus controlling the location of the plaque-treating device when extended from the distal end of the positioning catheter's lumen.

### CROSS-REFERENCE TO RELATED APPLICATIONS

This application claims the benefit of U.S. Provisional Application Ser. No. 61/606,655, filed Mar. 5, 2012. The disclosure of the prior application is considered part of (and is incorporated by reference in) the disclosure of this application.

### TECHNICAL FIELD

This document relates to treating occlusions within body vessels, for example fully occluded body vessels.

### BACKGROUND

Many people suffer from lack of vascular supply, for example to the lower extremities. In fact, in some cases people may have total occlusions of a body vessel, for example in the peripheral arterial system for patients suffering from peripheral arterial disease. Total occlusions may consist of different kinds of substances causing the occlusion within the artery. For example, certain portions of the arterial system may be occluded with soft material or with atheroma. Other areas may have hardened calcific plaque which is not easy to penetrate.

Various devices are known for treating body vessels containing soft material and hardened calcific plaque. Such devices include rotational atherectomy devices that include a rotational burr element that is able to break up or free hardened plaque formed on a vessel wall. Devices such as guidewires have also been used to push through softer materials contained within the vessel and thereby create a more open passageway within the vessel. Treating vascular occlusions is made challenging when there is variability in the plaque morphology, and when occlusions are located at or near a curvature in a vessel. In some cases, it is possible through imaging techniques for a medical practitioner to visualize the passage that is to be traversed; in other cases the path cannot be visualized clearly.

### SUMMARY

This document describes, in one aspect, systems and methods for treating occlusions within body vessels. Generally, such a system includes a lumen-forming device, for example a rotational atherectomy device, having an elongate shaft and a distal plaque-treating portion, for example a burr element, adapted to dislodge hardened plaque from within a body vessel. The system also includes a multi-balloon positioning catheter with a lumen into and through which lumen the plaque-threating device may be extended. The positioning catheter has at least three inflatable balloons positioned circumferentially about the lumen to controllably position the lumen and its distal opening at a desired location and trajectory within the body vessel. As such, the positioning catheter controls the location and trajectory of the plaque-treating device when extended from the distal end of the positioning catheter's lumen.

In another aspect, this document describes a method for treating deposits from a body vessel. The method includes introducing a positioning catheter into a body lumen. In this method, the positioning includes a lumen into and through which lumen a deposit-treating device is extendable. The positioning catheter further includes at least three inflatable balloons positioned circumferentially about the positioning catheter lumen. The method also includes controlling an inflation level of each of the at least three circumferentially positioned inflatable balloons to position the lumen and its distal end at the desired location and trajectory within the body vessel, introducing the deposit-treating device into the lumen until its deposit-treating distal portion extends distal of a distal opening of the positioning catheter, and treating deposits from within the vessel using the deposit-treating device.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram of an example system for treating deposits from within a body vessel, wherein in this embodiment the system includes a positioning catheter, a rotational atherectomy device, and an external handle/control system.

FIGS. 2A and 2B are, respectively, perspective and cross-sectional diagrams of an example embodiment of a distal portion of a positioning catheter.

FIG. 3 is cut-away diagram showing an example embodiment of a distal portion of a positioning catheter with a rotational atherectomy device extending through a lumen in the positioning catheter. The figure further illustrates the supply of lubricant (for example, Rotaglide) and/or cooling solution.

FIGS. 4A and 4B are, respectively, side view and cross-sectional diagrams of an example embodiment of a distal portion of a rotational atherectomy device, wherein, and shown in hidden lines, the rotational atherectomy device has a central longitudinal guidewire lumen extending through the device for the introduction of a guidewire into and through the rotational atherectomy device.

FIGS. 5A-5C are diagrams showing an example method of using the system for treating deposits from within a body vessel.

FIG. 5D is a diagram showing an example method of using the system within a curved body vessel.

FIGS. 6A-C are diagrams of an example system wherein the positioning catheter has two sets of three inflatable balloons, with one set being distal of the other.

FIG. 7 is a diagram of the FIG. 6 embodiment being used in a curved vessel. Like reference symbols in the various drawings indicate like elements.

### DETAILED DESCRIPTION

### Systems and Devices Description

The systems and devices described in this document and shown in the accompanying figures are designed to traverse and treat total occlusions, for example in the peripheral arterial system for patients suffering with peripheral arterial disease. One example patient population that may be served by the systems and devices are people suffering from a lack of vascular supply to the lower extremities. In this example, total occlusions in this patient population may consist of different kinds of substances causing the occlusion within the artery. For example, certain portions of the arterial system may be occluded with soft material such as atheroma. Other areas may contain hardened calcific plaque which is not easy to penetrate. In some embodiments, the systems and devices allow crossing of these peripheral occlusive segments of the arterial system so that recanalization can be performed to allow vascular supply to the lower extremity. Other embodiments of the systems and devices may be applied to other arterial circulations within the body, for example recanalization of occluded arteries in the heart.

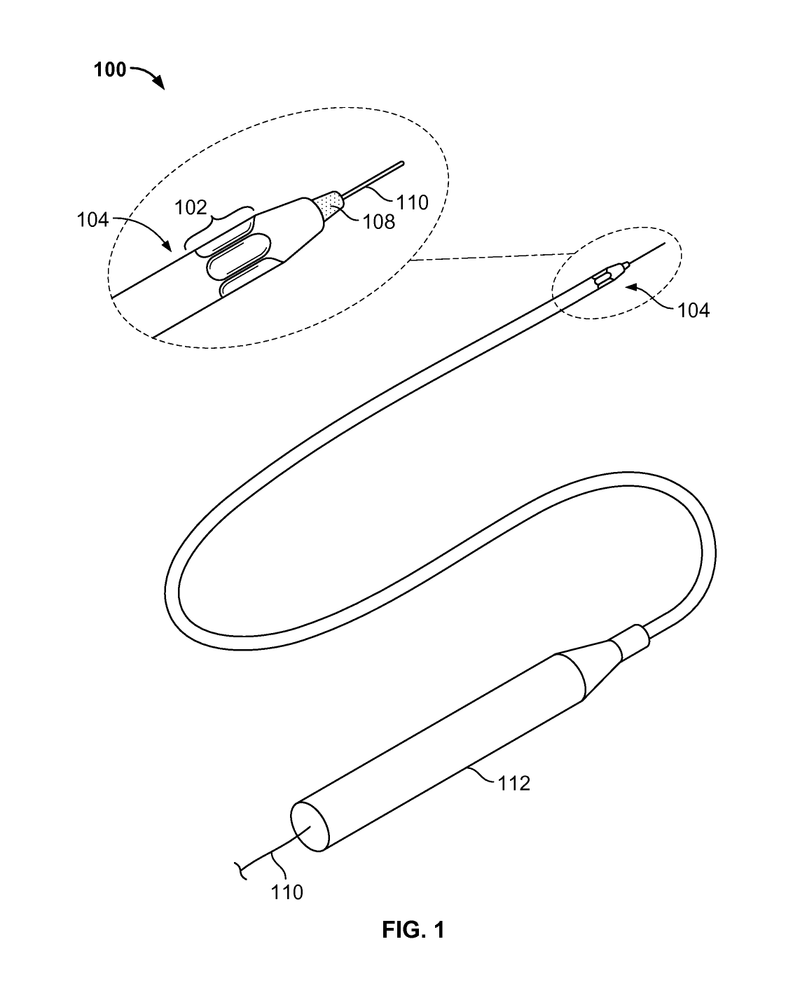

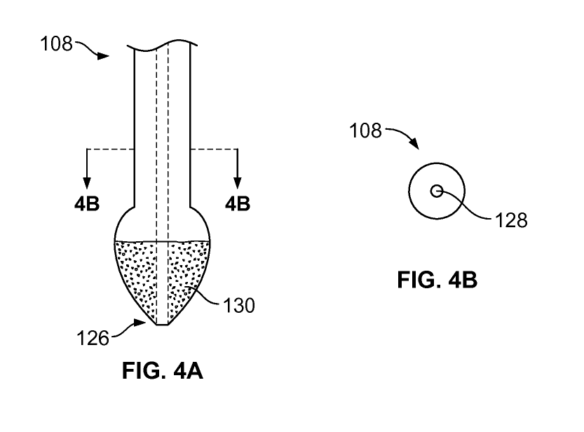

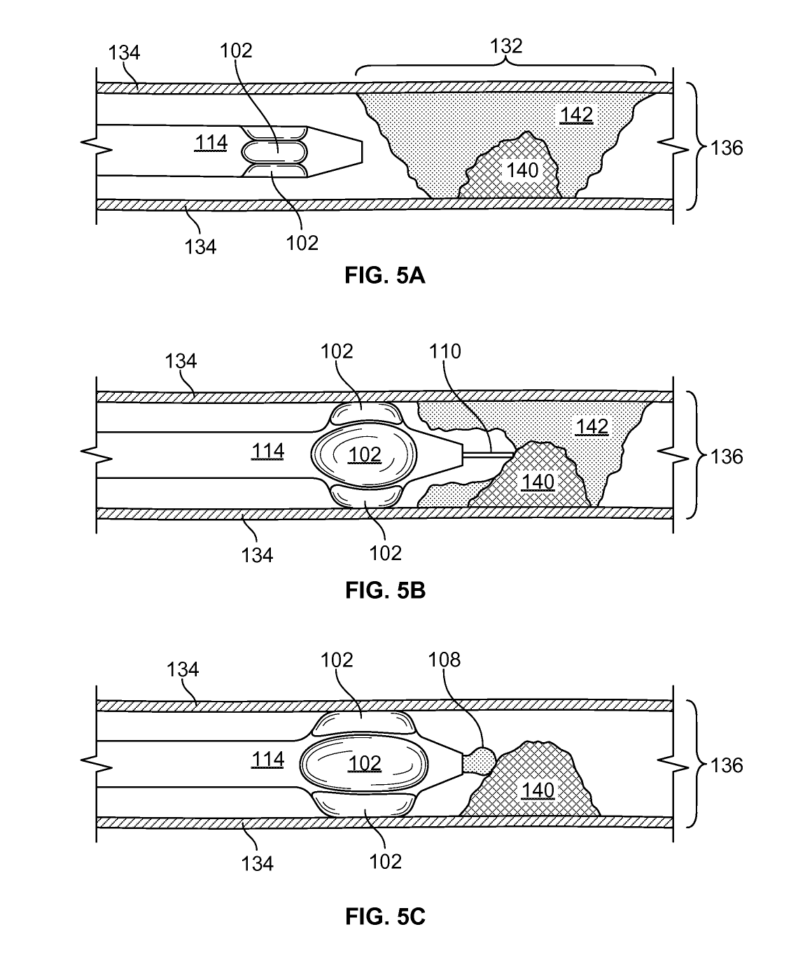

FIGS. 1-4 illustrate one embodiment of an example device 100 that includes one set of three equal-sized inflatable balloons 102 that are positioned around the entire periphery of a positioning catheter 104. The device 100 can be part of a system, for example, for treating deposits from within a body vessel. Referring to FIG. 1, the device 100 includes the three balloons 102, the positioning catheter 104, a plaque-treating device (e.g. a rotational atherectomy device 108), a guidewire 110, and an external handle/control system 112. Other embodiments of the device 100 can include more than three balloons 102 in various arrangements, such as four or more balloons in a proximal set positioned around a circumference of the positioning catheter 104, and can also include other components in addition to those shown for the device 100. Further, as described below, there can be multiple proximal sets of balloons 102. In some implementations, the positioning catheter 104 can be made of a material, such as plastic, metal or other materials, such that the balloons 102 can be effective in positioning the positioning catheter 104 for effective operation by a medical practitioner.

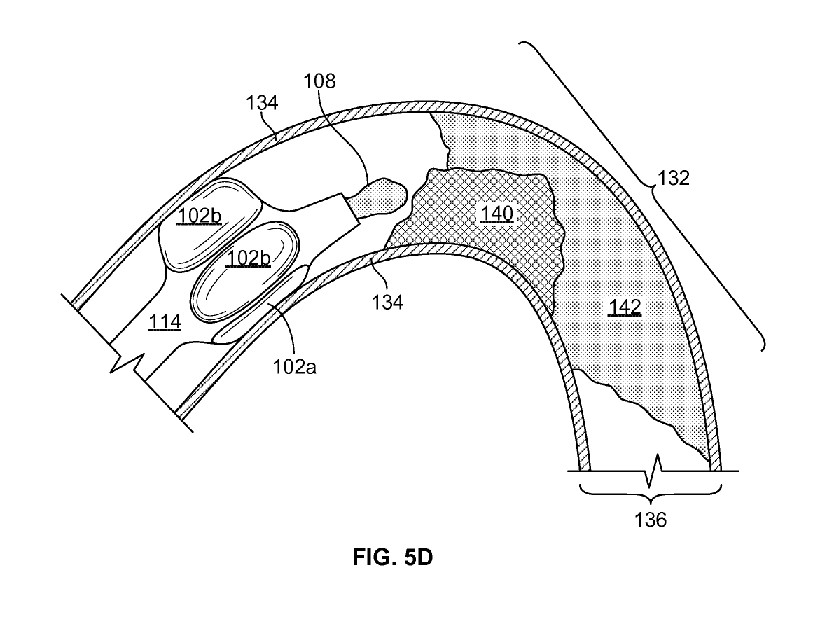

FIGS. 5A-5C provides a series of diagrams illustrating how the device 100 shown in FIGS. 1-4 may be operated to treat an occluded vessel, such as a total occlusion 132. Importantly, the device 100 provides control that keeps the rotational atherectomy device 108 in the center of a lumen 122 within the device 100, and prevents the rotational atherectomy device 108 from being deflected axially such that it may be inadvertently directed into a vessel wall 134, causing damage. The three balloons 102 may be inflated or deflated as a group, or each balloon 102 may be independently controlled, depending on the desired trajectory of the plaque-treating device (e.g., the rotational atherectomy device 108) that is desired. Independent control of each of the balloons 102 assists in achieving a proper trajectory of the plaque-treating device when addressing a curve in a vessel.

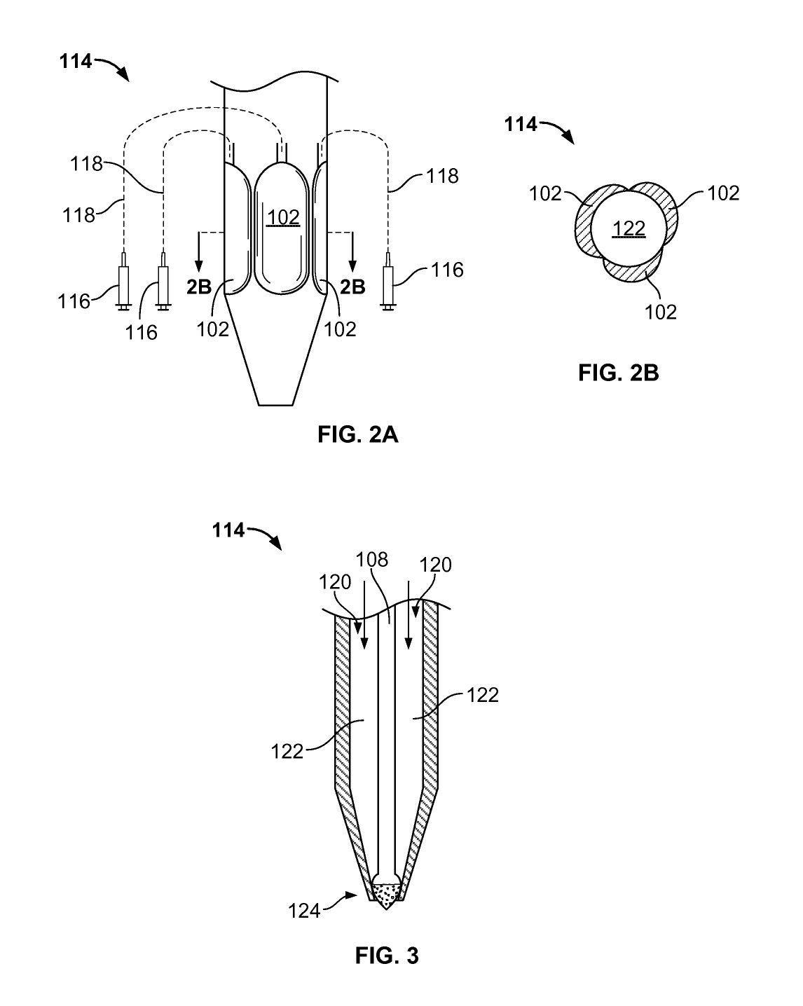

FIGS. 2A and 2B are, respectively, perspective and cross-sectional diagrams of an example embodiment of a distal portion 114 of the positioning catheter 104. In some implementations, pumps 116 connected to pump ends of inflation lines 118 that are connected at balloon ends to balloons 102, can be used to variably inflate the balloons 102. For example, each balloon 102 can be variably inflated to control the position of a tip 124 on the distal portion 114.

In some implementations, the device 100 includes a control device adapted to control inflation of the circumferentially positioned inflatable balloons to position the lumen at the desired location within the cross-section of the body vessel. For example, using the control device, a medical practitioner can inflate the balloons 102, e.g., using the pumps 116 that are included with or connected to the control device. In some implementations, the control device is adapted to provide lubricant 120 into the positioning catheter lumen 122 for supply of the lubricant 120 to the distal plaque-treating portion 114.

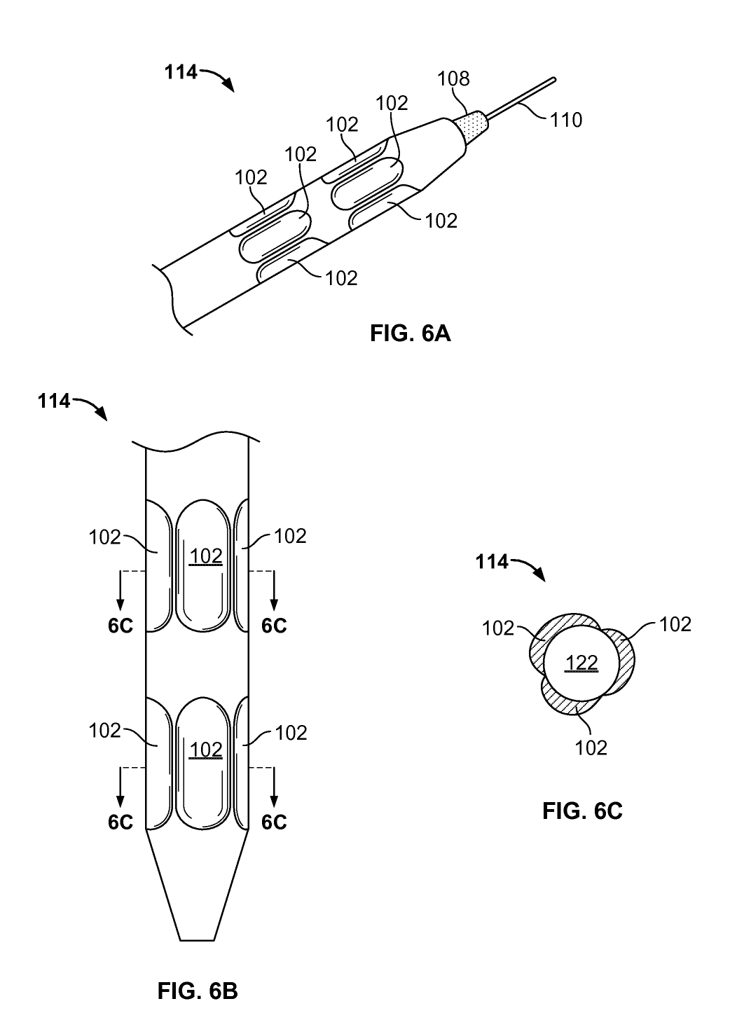

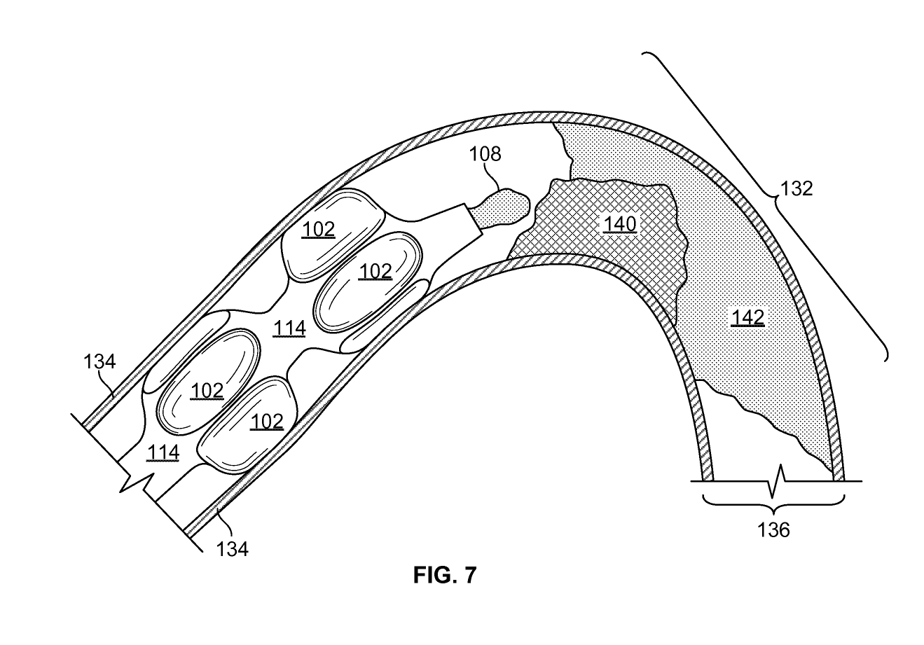

FIG. 6 illustrates another embodiment of the device 100 that includes two sets of three equal-sized inflatable balloons 102, wherein each set of three balloons 102 is positioned around the entire periphery of the positioning catheter 104. This embodiment may have an advantage of providing further control in maintaining the centering of a plaque-treating device (e.g., the rotational atherectomy device 108) when addressing a situation where plaque is contained at or near a curvature in a vessel. In this case, all six balloons 102 may be independently controllable. Use of the FIG. 6 embodiment in this manner is illustrated, for example, in FIG. 7.

In one embodiment applicable at least to treat occlusions of peripheral arteries, a positioning catheter 104 will be of a smaller caliber, for example #4 French to #5 French in size. In addition, the outside of the positioning catheter 104 may be coated with lubricant 120 (e.g., lubricious “glide” coating) to allow easy passage through the occluded segment. The lubricant 120 may be supplied through the lumen 122 in the positioning catheter 104.

As shown in the figures, the positioning catheter 104 includes the lumen 122 allowing passage through the positioning catheter 104 of the plaque-treating device, such as the rotational atherectomy device 108 or another device that delivers ultrasonic vibrations or laser energy to a distal portion of the plaque-removal device. The lumen 122 of the positioning catheter 104 may further be sized so that the positioning catheter 104 may allow passage of the rotational atherectomy device 108, as well as lubricant 120 to bathe the device beyond its distal tip 124. The lumen 122 of the positioning catheter 104 may also allow passage of the guidewire 110, for example a 0.035 guidewire. In addition or alternatively, the guidewire 110 may be introduced through a lumen 122 in the rotational atherectomy device 108.

The distal portion of the positioning catheter 104 has, in one embodiment, three inflatable balloons 102. The balloons 102 will serve as a mechanism to allow directional movement of a tip 126 of the rotational atherectomy device 108 within the cross-section of the body lumen 128, as well as providing a locking mechanism that allows stabilization of the catheter tip just above a total occlusion.

The three inflatable balloons 102 may be, as shown in the figures, of similar size to one another and positioned around the entire circumference of the lumen 122 of the positioning catheter 104. In other embodiments, there may be more than three balloons 102, and they may be sized differently from one another. Each of the positioning balloons 102 may be controlled independently. The balloons 102 will serve as a mechanism to allow directional movement of the tip within the cross-section of the body lumen 122, as well as providing a locking mechanism that allows stabilization of the catheter tip just above a total occlusion.

### Methods of Utilization

In use, the positioning catheter 104 may be passed down an arterial sheath that is placed above a total occlusion. Once the positioning catheter 104 is in position at the site of total occlusion, the guidewire 110 (for example, a 0.035 French guidewire) may be used to loosen soft deposits 142 (see FIG. 5B). The medical practitioner may sense the presence of hardened plaque 140 by feel (tactile feedback and interpretation by a skilled operator) when manipulating the guidewire 110 to loosen soft deposits 142. The positioning catheter 104 may be incrementally moved distally during the procedure, as the medical practitioner works a path distal through the vessel.

At a location where the presence of hardened plaque 140 is detected, for example by feel, the medical practitioner may advance the positioning catheter 104 to be near the site of the hardened plaque 140 (see FIG. 5C), and may equally inflate each of the three balloons 102 in a case where the occlusion is in a relatively straight vessel as shown in FIG. 5C. After the positioning catheter 104 is so positioned with the three circumferential balloons 102 equally inflated, the guidewire 110 may be pulled proximally (retracted), and the rotational atherectomy device 108 advance out of the distal tip of the positioning catheter 104. Owing to the equal inflation of the three balloons 102, the rotational atherectomy device 108 will be centered in the vessel, and will be directed in a straight head-on trajectory. As illustrated in FIG. 5C, the balloons 102 stabilize the trajectory, and prevent the balloon 102 from deflecting when the rotational atherectomy device 108 comes in contact with hardened plaque 140, which deflection may cause damage to a vessel wall 134 into which the rotational atherectomy device 108 may become otherwise deflected.

The rotational atherectomy device 108 can be positioned in a heavily calcified plaque and turned on allowing ablation of the calcific plaque and the creation of a small, for example 1 mm or less, orifice or hole through the calcified plaque. The rotational atherectomy device 108 may be moved forward in small increments using the distal portion of the positioning catheter 104 as well as a 0.035/0.014/0.018 wire that advances through and can operate to guide the rotational atherectomy device 108.

At the sites of total occlusion, the device 100 can be turned on without fear of perforation as the tip of the rotational atherectomy device 108 is stabilized by the catheter which is directly above it (the catheter is locked into position by inflating the balloons 102 which oppose the walls of the lumen of the artery 136).

As shown in FIG. 5D, which shows the system of FIGS. 1-4 in a curved vessel, it may be desirable in these circumstances to inflate the three balloons 102 to different levels. For example, balloon 102a on the inside of the curve may be inflated less than balloons 102b on the outside of the curve, or not at all. In this manner, the rotational atherectomy device 108 is no longer positioned within the center of the vessel, but rather is positioned closer to the inside of the curve. As such, given that the potential for a damaging deflection is greater in a curved vessel, such a technique provides further safety from a damaging deflection.

Referring now to FIG. 7, there is shown an example use of the FIGS. 6A-6C system in a curved vessel. As shown in FIG. 7, the distal set of balloons 102 are inflated such that a balloon 102 on the outside of the curve is inflated to a greater level than a balloon 102 positioned on the inside of the curve. Concurrently, for the proximal set of balloons 102, a balloon 102 positioned on the inside of the curve is inflated to a greater level than the other balloons 102. In this manner, the rotational atherectomy device 108 not only achieves a positioning within the vessel that is more toward the inside of the vessel curve than the outside of the vessel curve, but also the rotational atherectomy device 108 is given a trajectory that is oriented around the curve of the vessel. Such a trajectory in a curved vessel may help provide further protection from a potentially damaging deflection of the rotational atherectomy device 108.

### Control of the Positioning Catheter

Control of the positioning catheter 104 may be achieved by use of a control unit that may be provided with the system. The control unit may provide a visual display to the operator through the procedure. The control unit may include actuation mechanisms (such as a joystick, buttons, etc.) that receive inputs from the operator to control the inflatable balloons 102 in a desired manner. The control unit may include a processing component that receives the user input, calculates an appropriate inflation level for each of the balloons 102, and provides an output signal to actuation mechanisms that inflate or deflate each of the balloons 102 to the appropriate calculated level.

### Additional Features of an Example Rotational Atherectomy Device

The shaft of the rotational atherectomy device 108 may be strengthened so as not to buckle in its distal 2 cm. The rotational atherectomy device 108 may have a cutting tip estimated to be one-tenth of a millimeter in diameter. The forward half of the rotational atherectomy device 108 may be covered with industrial diamonds 130 that will allow ablation of calcific plaque. The rotational atherectomy device 108 may include a lumen to allow passage of said guidewire 110. All the above equipment may be radiopaque to allow visualization under fluoroscopic guidance.

A number of embodiments of the invention have been described. Nevertheless, it will be understood that various modifications may be made without departing from the spirit and scope of the invention. Accordingly, other embodiments are within the scope of the following claims.

What is claimed is:

1. A system for treating occlusions present within a body vessel, the device comprising:
a plaque-treating device having an elongate shaft and a distal plaque-treating portion adapted to remove hardened plaque from within a body vessel; and
a multi-balloon positioning catheter with a lumen into and through which lumen the plaque-treating device may be extended, the positioning catheter having at least three inflatable balloons positioned circumferentially about the lumen to controllably stabilize the lumen and its distal opening at a desired location within the body vessel, thus controlling the location of the plaque-treating device when extended from the distal end of the positioning catheter's lumen.

2. The system of claim 1, wherein the plaque-treating device comprises a rotational atherectomy device.

3. The system of claim 2, wherein the rotational atherectomy device comprises a burr element.

4. The system of claim 1, wherein the plaque-treating device comprises a guidewire lumen extending therethrough.

5. The system of claim 4, further comprising a guidewire sized for introduction into and through the guidewire lumen such that the guidewire is advanceable distally such that a distal portion of the guidewire extends distal of the plaque-treating device when needed.

6. The system of claim 1, wherein the circumferentially positioned inflatable balloons are sized and adapted to lock the positioning catheter at a desired location within the body vessel.

7. The system of claim 1, wherein the circumferentially positioned inflatable balloons are positioned near a distal end of the positioning catheter.

8. The system of claim 1, wherein the circumferentially positioned inflatable balloons are independently inflatable to position the lumen at the desired location within the cross-section of the body vessel.

9. The system of claim 1, further comprising a control device.

10. The system of claim 9, wherein the control device is adapted to control inflation of the circumferentially positioned inflatable balloons to position the lumen at the desired location within the cross-section of the body vessel.

11. The system of claim 9, wherein the control device is adapted to provide lubricant into the positioning catheter lumen for supply of the lubricant to the distal plaque-treating portion.

12. The system of claim 11, wherein the device is sized and adapted for use in a peripheral artery application.

13. A method for treating deposits from a body vessel, the device comprising:
introducing a positioning catheter into a body lumen, wherein the positioning catheter comprises:
a lumen into which a deposit-treating device is extendable, and at least three inflatable balloons positioned circumferentially about the lumen;
controlling an inflation level of each of the at least three circumferentially positioned inflatable balloons to position the lumen and a distal opening of the lumen at a desired location and trajectory within the body vessel;
introducing the deposit-treating device into the positioning catheter lumen until at ii least a portion of the deposit-treating distal portion extends out of the distal opening of the positioning catheter; and
treating deposits from within the vessel using the deposit-treating device.

14. The method of claim 13, wherein the deposit-treating device comprises a plaque-treating device.

15. The method of claim 14, wherein the plaque-treating device comprises a rotational atherectomy device.

16. The method of claim 14, further comprising advancing a soft-deposit-treating device through a lumen in the plaque-treating device, and treating soft deposits from within the vessel using the soft-deposit-treating device.

17. The method of claim 16, wherein the soft-deposit-treating device comprises a guidewire.

18. The method of claim 13, wherein the deposit-treating device comprises a guidewire.
